(19) United States
(12) Patent Application Publication
(10) Pub. No.: US 2023/0383241 A1
(43) Pub. Date: Nov. 30, 2023

(54) RECOVERY OF PERFUSION BLEED PRODUCT VIA ALTERNATING TANGENTIAL FLOW FILTRATION IN A SEDIMENTATION REACTOR

(71) Applicant: Repligen Corporation, Waltham, MA (US)

(72) Inventors: Charles Hill, Groton, MA (US); Joey Tse, San Jose, CA (US); Mario Sinani, Newton Upper Falls, MA (US); Earl Pineda, Anglet (FR)

(73) Assignee: Repligen Corporation, Waltham, MA (US)

(21) Appl. No.: 18/200,112

(22) Filed: May 22, 2023

Related U.S. Application Data
(60) Provisional application No. 63/365,283, filed on May 25, 2022.

(51) Int. Cl. C12M 1/00

(52) U.S. Cl. CPC C12M 47/10; C12M 29/10

(57) ABSTRACT

A bleed recovery system for use in a bioreactor system is disclosed. The bleed recovery system being configured to recover product of interest from the bleed material of the bioreactor system. In one embodiment, the bioreactor system includes a bleed recovery system for removing cell cultures from the bioreactor. The bleed recovery system includes a bleed pump coupled to the bioreactor so that cell bleed material can be removed from the bioreactor, a bleed vessel for receiving the cell bleed material, a cell retention device coupled to the bleed vessel, a harvest pump coupled to the cell retention device to deposit the product of interest into a second harvest tank, and a bleed pump connected to the bleed vessel to deposit the spent bleed material into a waste tank. Thus arranged, some or all of the product of interest can be separated from the spent medium.

### CROSS-REFERENCE TO RELATED APPLICATIONS

This is a non-provisional of pending provisional patent application Ser. No. 63/365,283, filed on May 25, 2022, the entirety of which application is incorporated by reference herein.

### FIELD

The present disclosure relates generally to the field of bioreactor systems and methods of harvesting a cell product from a cell culture by culturing cells in a fluid medium until the cells have produced a cell product at a harvest concentration. More particularly, the present disclosure relates to an improved bleed recovery system and method for recovering product of interest from the bleed (e.g., discarded, removed, etc.) medium or material of the bioreactor system.

### BACKGROUND

As will be appreciated by one of ordinary skill in the art, bioreactor systems are known for cultivating cell cultures. Cultures of microbial, plant, or animal cells may be used to produce biological and chemical substances of significant commercial value. Particularly for commercial production, these cultures can be run in four operational modes: batch, continuous “chemostat”, fed-batch, or cell retention. Applications include fermentation, biotechnology, and chemical, for production of specialty chemicals and products, as well as waste-treatment. The products are typically high-value products that include any desired cellular products, such as endogenous and recombinant products, including proteins, peptides, nucleic acids, virus, amino acids, antibiotics, specialty chemicals and other molecules of value. Desired proteins may include but are not limited to monoclonal antibodies, enzymes and other recombinant antibodies, enzymes, peptides, virus. Even marginal improvements in yield and productivity increase profitability. Therefore, there are incentives to improve batch, continuous, fed-batch, or cell retention reactor operations.

In use, a bioreactor system using a perfusion culture uses a cell retention device to continuously replenish cell culture media, remove waste products, and harvest product, while retaining the cells within the bioreactor system. A steady-state perfusion process at a minimum includes a flowrate of fresh media in, cell free harvest out, and a bleed of cell culture out to maintain cells at a target density (e.g., bleed removes cell cultures from the bioreactor to establish a steady-state operation of bioreactor culture by keeping viable cell density (VCD) constant). Bleed operations can be conducted semi-continuously, on a daily basis, or continuously, via online biomass probe or daily adjustment from offline measurement, to maintain the target cell density. Typically, this bleed stream is wasted with no recovery of the product of interest. The bleed is discarded with no recovery of targeted product. A good perfusion process operates around 5-10% of the bioreactor bled per day with some processes exceeding that.

Others have tried to reduce bleed flow rates via external environmental levers (temperature, pH, osmolality) to block cells in G0/G1 state to inhibit cell growth and reduce bleed/product lost. However, these methods can have unwanted side effects on culture duration and product quality meaning there is a need for a mechanism to recover lost product from bleed without altering the culture environment.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various features of the present disclosure, a system and/or method of harvesting product of interest from a bleed of a perfusion bioreactor is disclosed. In one embodiment, the system for harvesting product of interest from a bleed material of a perfusion bioreactor includes a bioreactor arranged and configured to store a medium including a product of interest; a first cell retention device coupled to the bioreactor via tubing, the first cell retention device arranged and configured to separate the product of interest from the medium; a first harvest pump coupled to the first cell retention device via tubing to transfer the product of interest from the first cell retention device to a first harvest tank; and a bleed recovery system coupled to the bioreactor via tubing, the bleed recovery system arranged and configured to receive the bleed material including the product of interest. In one embodiment, the bleed recovery system includes a first bleed pump operatively coupled to the bioreactor via tubing; a bleed vessel connected the first bleed pump, the bleed vessel arranged and configured to receive the bleed material including the product of interest from the first bleed pump; a second cell retention device coupled to the bleed vessel via tubing, the second cell retention device arranged and configured to receive the product of interest; a second harvest pump coupled to the second cell retention device via tubing to transfer the product of interest from the second cell retention device to a second harvest tank; and a second bleed pump connected to the bleed vessel to transfer the medium to a bleed waste tank.

In one embodiment, the system collects bleed material from the perfusion bioreactor in the bleed vessel and harvests product of interest through the second harvest pump.

In one embodiment, the bleed vessel may be in the form of a sedimentation style bleed vessel.

In one embodiment, the flowrate of Bleed 1 (e.g., flowrate of the first bleed pump exiting the bioreactor) equals the flowrate of Bleed 2 (e.g., flowrate of the second bleed pump exiting the bleed vessel) plus the flowrate of Harvest 2 (e.g., flowrate of the material exiting the second cell retention device coupled to the bleed vessel).

In some embodiments, the bleed collection (e.g., flowrate of the first bleed pump exiting the bioreactor) can be semi-continuous, constantly continuous, or dynamically continuous. In certain embodiments, the bleed rate can be between 1 to 50% of the bioreactors vessel volume per day (VVD).

In particular embodiments, the flowrate of the first bleed pump exiting the bioreactor equals the difference between media-in (e.g., the flowrate of new media being pumped into the bioreactor) and harvest 1 (e.g., the flowrate of the first harvest pump exiting the first cell retention device coupled to the bioreactor).

In some embodiments, the Harvest 1 pump (e.g., the first harvest pump coupled to the exit port of the first cell retention device coupled to the bioreactor) can be semi-continuous, constantly continuous, or dynamically continuous. In certain embodiments the rate can be between 0.25 and 5 VVD or 0.01 to 1 nL/cell/day.

In some embodiments, the Bleed 1 flowrate (e.g., flowrate of the first bleed pump exiting the bioreactor) can be calculated offline or online cell density data. In certain embodiments, the Bleed 1 rate (e.g., flowrate of the first bleed pump exiting the bioreactor) is increased or decreased to maintain a target cell density. In certain embodiments, the target cell density can be between 10e6 and 300e6 cells/mL.

In some embodiments, the Bleed 2 pump (e.g., flowrate of the second bleed pump exiting the bleed vessel) and Harvest 2 pump (e.g., flowrate of the second harvest pump exiting the bleed vessel) can be operated semi-continuously, constantly continuous, or dynamically continuous. In particular embodiments, the flowrate of Bleed 2 (e.g., flowrate of the second bleed pump exiting the bleed vessel) equals the flowrate of Bleed 1 (e.g., flowrate of the first bleed pump exiting the bioreactor) minus the flowrate of Harvest 2 (e.g., flowrate of the second harvest pump exiting the bleed vessel). In certain embodiments, Bleed 2 and Harvest 2 can start once the liquid level in the bleed vessel reaches the inlet of the cell retention device.

In some embodiments, the perfusion bioreactor is a production perfusion bioreactor. In some embodiments, the perfusion and bleed bioreactors are attached to an alternating tangential flow system or a tangential flow system.

In some embodiment, the cell retention devices are one of an alternating tangential flow (ATF) filtration device or a tangential flow filtration (TFF) device. Alternatively, in some embodiments, a tangential flow depth filtration device (TFDF) may be used.

In some embodiments, the bioreactor is stainless steel, plastic, or glass.

In some embodiments, the bioreactor is a stirred tank bioreactor. In some embodiments, the bioreactor is a rocker bioreactor. In some embodiments, the bioreactor is an orbitally shaken bioreactor.

In some embodiments, the alternating tangential flow or tangential flow system incorporates a hollow fiber filter with a certain size cutoff. In certain embodiments, the size cutoff is 0.1 to 0.65 μm. In certain embodiments, such as in TFDF devices, the size cutoff is 2 to μm. In some embodiments, the alternating tangential flow or tangential flow system incorporates a hollow fiber filter with a molecular weight cutoff depending on product size (1-750 kDa).

In some embodiments, the tangential flow device incorporates a pump for cross flow. In certain embodiments this crossflow rate is 0.1 to 80 LPM.

In some embodiments, one or more cell retention devices (e.g., the ATF, TFF, or TFDF device) could be used in a single production vessel.

In some embodiments, the bleed vessel is 0.25-2000 L in working volume.

In some embodiments, the bleed vessel is stainless steel, plastic, or glass.

In some embodiments, the pumps are peristaltic, diaphragm, or magnetic levitating.

In certain embodiments, the perfusion process can run for 6-60 days.

In some embodiments, the cells are mammalian. In other embodiments, the cells are non-mammalian.

In some embodiments, the product of interest is a polypeptide. In other embodiments, the product of interest is a virus. In other embodiments the product of interest is a viral vector.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an embodiment of a bioreactor system including a bleed recovery system in accordance with one or more features of the present disclosure; and

FIG. 2 illustrates an alternate embodiment of a bleed recovery system that may be used in the bioreactor system of FIG. 1 in accordance with one or more features of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more features of this disclosure. Each example of an embodiment is provided by way of explanation and is not the only way to implement these features but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed features will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

The present disclosure describes systems and/or methods of recovering or harvesting product of interest from the bleed of a perfusion bioreactor system.

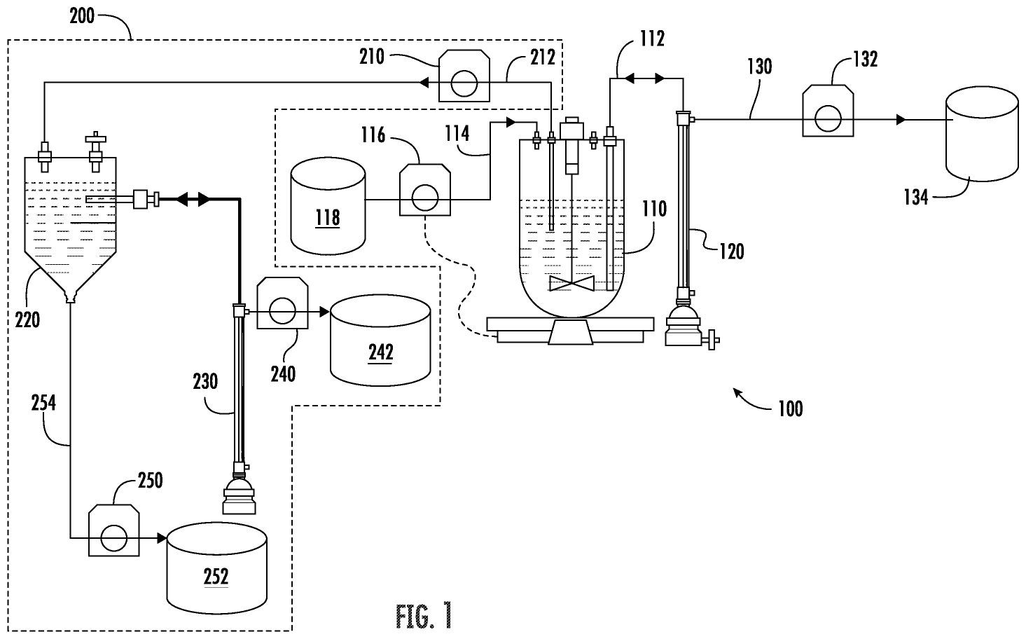

As will be appreciated by one of ordinary skill in the art, with reference to FIG. 1, in use, a bioreactor system 100 such as, for example, an alternating tangential flow (ATF) harvest bioreactor system, is arranged and configured to include a cell retention device to continuously replenish cell culture media, remove waste products, and harvest product, while retaining an appropriate level of cells within the bioreactor system. For example, as illustrated in FIG. 1, the bioreactor system 100 includes a tank, a vessel, a bioreactor, or the like 110 (terms used interchangeably herein), which can be, for example, a stirred tank reactor. The bioreactor 110 is connected via tubing 112 (e.g., drain tubing) to a cell retention device 120 such as, for example, an ATF device. The ATF device 120 is a system such as ones used to perfuse a bioreactor culture using hollow fiber filtration using alternating tangential flow. The ATF device 120 includes a device that controls a diaphragm pump to perform ATF through a hollow fiber filter (see, e.g., U.S. Pat. No. 6,544,424) both of which may be encased in a sterilizable housing.

In use, as will be readily appreciated by one of ordinary skill in the art, medium and additives are introduced into the bioreactor 110 via a feed line 114 coupled to a fresh media tank 118, which is controlled by a valve and/or media pump 116. In use, fresh medium is pumped from the fresh media tank 118 to the bioreactor 110. In addition, medium and additives are removed from the bioreactor 110 via tubing 112 and transferred to the ATF device 120. The ATF device 120 includes a housing, a pump (e.g., a diaphragm pump), and a filter (e.g., a hollow fiber filter). In use, an air and vacuum supply source and a controller may be connected to the pump (e.g., diaphragm pump) of the ATF device 120 via an air tube. In use, air may be added and withdrawn from the diaphragm pump so as to increase and decrease the volume of the chambers contained within the diaphragm pump, altering the pressure within the housing of the ATF device 120 and directing flow of the fluid contained within the housing and drawing fluid across the membrane of the hollow fiber filter. Typically, the interior portion of the hollow fibers is fluidly connected to the bioreactor 110 via tubing 112 while the chamber outside the hollow fibers of the hollow fiber filter and within the housing is fluidly connected to tubing 130 (e.g., product or harvest drain tube). The tubing 130 has a harvest pump/valve 132 that controls withdrawal of the products that filter across the hollow fiber filter and reside in the chamber between the hollow fiber filter and housing. In use, the filtered product (e.g., product of interest) is deposited in a harvest tank 134 (e.g., also referred to herein as a first harvest tank). In FIG. 1, the tubing 130 is shown near the top of the ATF device 120, however the tubing 130 could also be located near the middle or bottom of the ATF device 120. Alternatively, there may be more than one tubing 130 connected to the housing of the ATF device 120.

In some embodiments, to carry out product harvesting using the ATF harvest bioreactor system 100, the product (e.g., the protein, recombinant proteins, monoclonal antibodies, vaccines, viral vectors, etc.) is harvested using ATF. The rapid harvest can be accomplished by cyclical removal of volume from the bioreactor 110 and refilling (batch filtration) or by continually replenishing the liquid in the culture broth while harvesting liquid through the filtration process (constant volume diafiltration).

Additional information on an example of an embodiment of a bioreactor system is disclosed in U.S. patent application Ser. No. 15/659,562, filed on Jul. 25, 2017, entitled Alternating Tangential Flow Rapid Harvesting, the entire contents of which is incorporated by reference herein.

In accordance with one or more features of the present disclosure, an improved system and/or method for recovering or harvesting product of interest from the bleed material of the bioreactor system 100 is disclosed. That is, in use, the bioreactor system 100 also includes a bleed system for removing cell cultures from the bioreactor 110 in order to maintain a constant viable cell density or range of viable cell densities in the perfusion cell culture. In use, cell bleed or removal of cell cultures from the bioreactor 110 can be performed semi-continuous or continuous, and may be based on daily sampling of viable cell density (VCD) or online biomass sensors. Thus, cell bleed may be dependent on growth rate and target VCD. Cell densities that are too high can cause bioreactor control problems (e.g., limited bioreactor oxygen capacity, perfusion rate, and/or cell retention device). Typical range of bleed rate may be between 10 to 25 percent of bioreactor volume per day. However, bleed rate has been known to be as high as 70 percent.

One disadvantage of existing bleed systems is that the system and/or method remove cell cultures from the bioreactor 110 to establish “steady-state” operation of bioreactor culture by keeping viable cell density (VCD) constant. In use, the stream of cell bleed is discarded with no recovery of product of interest.

With reference to the FIG. 1, in accordance with one or more principles of the present disclosure, the bioreactor system 100 includes a bleed recovery system 200. As illustrated, in one embodiment, the bleed recovery system 200 includes a bleed pump 210 (e.g., also referred to herein as a first bleed pump) coupled to the bioreactor 110 via tubing 212 so that the cell bleed (e.g., spent medium) can be removed from the bioreactor 110. The bleed recovery system 200 also includes a cell bleed tank or bleed vessel 220. In use, the cell bleed (e.g., spent medium, which can also include product of interest) is pumped or transferred from the bioreactor 110 to the bleed vessel 220 via the bleed pump 210 and tubing 212.

In accordance with one or more features of the present disclosure, the bleed vessel 220 may be coupled to an alternating tangential flow (ATF) filtration or tangential flow filtration (TFF) device 230 connected horizontally or vertically to the bleed vessel 220 (referred to herein as a second ATF device), a harvest pump 240 (referred to herein as a second harvest pump) on the permeate side of the ATF or TFF device 230 to separate, collect, and/or deposit the product of interest into a second harvest tank 242, and a bleed pump 250 (referred to herein as a second bleed pump) connected to the bottom of the bleed vessel 220 to pump and deposit the bleed material (e.g., spent medium) into a waste tank 252. Thus arranged, in use and in accordance with one or more features of the present disclosure, some or all of the product of interest can be separated from the spent medium. Thereafter, the product of interest can be deposited in a storage tank while the spent medium can be discarded into a separate tank.

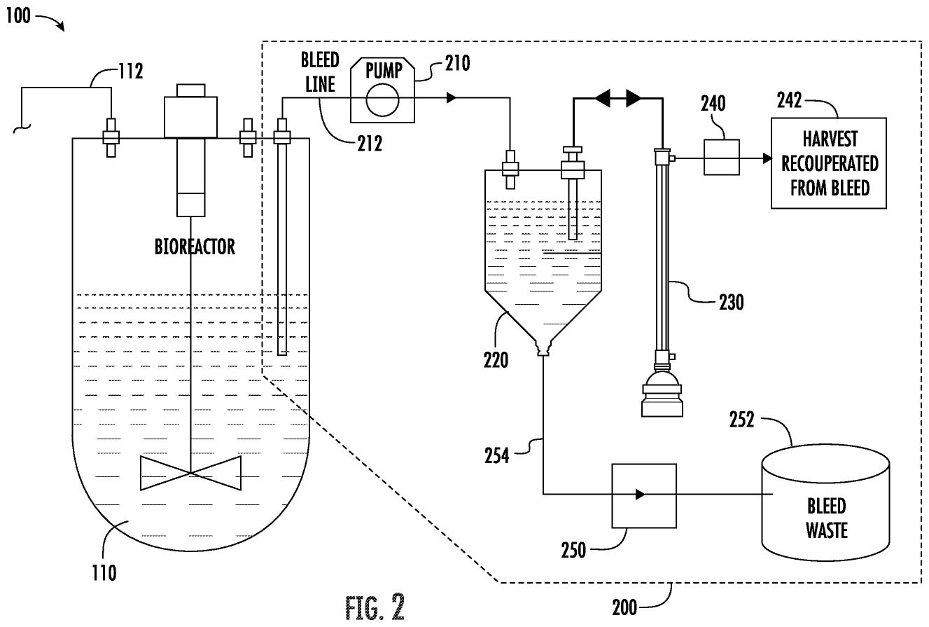

In one embodiment, as illustrated, the bleed vessel 220 may be in the form of a sedimentation tank. In one embodiment, the second bleed pump 250 may be coupled to tubing 254 that is coupled to a bottom portion of the sedimentation bleed vessel 220 so that any product and/or medium residing or depositing on the bottom of the bleed vessel 220 is removed or pumped via the second bleed pump 250 to the waste tank 252 via tubing 254. The ATF or TFF device 230 may be coupled to a side portion (as illustrated in FIG. 1) or a top portion (as illustrated in the alternate embodiment of FIG. 2) of the bleed vessel 220 so that product of interest positioned (e.g., floating) in the liquid adjacent to, or closer to, the top portion of the sedimentation bleed vessel 220 can be pumped or transferred to the second ATF device 230. Thus arranged, disturbance of the cell concentration or concentrate in the bleed vessel 220 is minimized.

As used herein, the term “cell culture” includes any combination of cells and medium. In various embodiments, the present disclosure uses the method of cell culture known as perfusion cell culture.

As used herein, the term “perfusion cell culture” or “perfusion” refers to the method of culturing cells, where fresh medium is added to the culture and spent medium is removed while cells are retained in the bioreactor. The fresh medium added provides additional nutrients that may have been depleted during the cell culture. The spent media is removed to reduce potential toxic byproducts and cellular waste. The removal of spent media can also remove the product of interest from the bioreactor. The perfusion process generally occurs during the growth phase and continues through the production of the product of interest.

As used herein, the rate at which spent media is removed is referred to as “perfusion rate”. Perfusion rate can either be quantified as a volumetric flow rate or in terms of “VVD” or “CSPR”. A VVD perfusion rate refers to the volume vessels exchanged per day. A CSPR perfusion rate refers to the perfusion rate based on cell density or perfusion rate/cell density. The perfusion rate can be constant, dynamic, or semi-continuous. The perfusion rate chosen depends on the cell line, growth rate, productivity, viable cell density, and other factors. The typical perfusion rate in VVD can range from 0.25 to 5. The typical perfusion rate in CSPR can range from 0.01 to 1.0 nL/cell/day. In the described experiment, the perfusion rate will be referred to as “Harvest 1” pump rate (e.g., flowrate of the first harvest pump 132 coupled to the first ATF device 120).

As used herein, the term “bleed” refers to the removal of cell culture (e.g., removal of spent medium including product of interest from the bioreactor) in order to maintain a constant viable cell density or range of viable cell densities in a perfusion cell culture. This can be done on a continuous basis by matching the bleed rate, “Bleed 1” rate (e.g., flowrate of bleed pump 210 coupled to the bioreactor) to the growth rate of the cell culture once it reaches the target cell density. The Bleed 1 rate can also be controlled by online measurements of cell density and can operate semi-continuously. The bleed can also be performed daily after offline sampling of cell density. The typical bleed range is between 0.05 and 0.1 VVD but can also be between 0.01 and 0.5 VVD.

As used herein, the rate at which fresh media is added to the culture is referred to as “Media In” (e.g., rate of media pump 116 or flowrate of pumping new media into the bioreactor). In order to maintain the volume in the bioreactor, the Media In rate should be set as Media In=Harvest 1 pump rate+Bleed 1 pump rate (e.g., flowrate of pumping new media into the bioreactor equals flowrate of the first harvest pump 132 coupled to the first ATF device 120 plus the flowrate of bleed pump 210 coupled to the bioreactor 110).

As used herein, the term “cell culture media” or “media” refers to the solution in which cells are grown. Cell culture media includes a variety of components such as amino acids, sugars, lipids, vitamins, trace elements, etc. These components provide the cell culture with a nutritional and physiochemical environment to promote growth and production of product. The cells used in the present disclosure can be cultured in any number of commercially available or in-house medias. Media selection is used to maximize cell growth, viability, and production of the product of interest.

As used herein, the term “cell density” refers to the number of cells in a given volume. Cell density can be monitored by taking a sample from the culture and analyzing under a microscope or commercial cell counting device. Cell density can also be monitored via commercially available biomass capacitance probes that output values correlated to cell density.

As used herein, the term “viable cell density” refers to the number of living cell present in a given volume and can also be referred to as “VCD”. The term “VCD max” refers to the viable cell density at which the bleed control will be used to prevent overgrowth of the cell culture. Typically, this value is optimized so that at a given perfusion rate and bioreactor condition, the cell culture remains healthy. The relation between VCD and total cell density is known as “cell viability”. Cell viability gives an indication of the cells ability to survive in current culture conditions. A VCD max setpoint is important to maintain cell viability.

As used herein, the term “cell” in the present disclosure refers to any cultured cells (mammalian, animal, insect, etc.) which can be grown in a media that provides the appropriate nutrients. In the present disclosure, the cells used are generally mammalian and express and secrete the product of interest.

As used herein, the term “growth phase” refers to the cell culture phase when a VCD at a given time is higher than the previous time point. During a perfusion culture, the cell culture will continue to grow until there are limitations of nutrients or other important physiological requirements run out. It is important in a perfusion process to set a VCD max that still allows growth. That growth will be countered with the bleed rate to maintain that VCD max by removing excess cells that will not be supported with the given culture conditions.

As used herein, the term “production phase” refers to the phase in cell culture when the cells produce the product of interest. This product of interest can be any therapeutic including monoclonal antibodies, polypeptides, virions, virus-like particles, DNA, RNA, etc. During the production phase of a perfusion cell culture, the bleed pump 210 not only removes cells but also some product of interest that cannot be further processed for therapeutic use.

As used herein, the term “bioreactor” refers to a closed container or vessel used to grow cells. The bioreactor also allows for the control important parameters for maintaining a healthy cell culture. Some of these parameters include pH, dissolved oxygen (DO), temperature, mixing through agitation, perfusion rate, media in, volume, and other critical parameters. In the present disclosure, any commercially available bioreactor, fermenter, or disposable reactor can be used. The volume of these bioreactors used in the present disclosure can range anywhere from 250 mL to 25,000 L depending on facility fit. Also, these bioreactors can be constructed of any material suitable for cell culture such as glass, plastic, or metal.

The cell culture grown in this present disclosure can be maintained at a temperature between 30° C. and 39° C. depending on what is appropriate for the cell type and culture conditions. In the present disclosure, the pH maintained in the bioreactor can range between 6.0 and 8.0. This range can be tightened and controlled by the bioreactor system through the addition of base, acid, or CO₂. The dissolved oxygen (DO) in the present disclosure can be maintained anywhere between 10% and 100% through the addition of O₂ and N₂ gas or through the increase and decrease of agitation.

In the present disclosure, the bioreactor may be equipped with a “cell retention device”, which refers to a device, internal or external to the reactor, that maintains cells in the bioreactor as spent media is removed. This device can be tangential flow filtration (TFF), alternating tangential flow (ATF), spin filter, ultrasonic separator, gravity settler, acoustic cell separator, continuous centrifuge, or any other device that retains cells. In the present disclosure, an ATF device is preferably used as a cell retention device.

In the present disclosure, the ATF device may include a hollow fiber filter to exchange media while retaining cells in the bioreactor. The ATF device may use a diaphragm pump that uses air and vacuum to pull the bioreactor contents through the hollow fiber filter and pushes it back into the bioreactor while permeate is being drawn across the filter with a separate pump. The rate at which the bioreactor contents are pulled through the hollow fiber filter can range anywhere between 0.1 and 80 LPM in the present disclosure.

As used herein, the term “bleed vessel” refers to a closed container or vessel that collects bleed material through the course of a perfusion cell culture. For the present disclosure, a sedimentation style bleed vessel may be used to support the separation of cells and product so that the bleed material can be further processed to capture lost product from the bleed. The bleed vessel may be filled at a rate equal to Bleed 1 rate (e.g., flowrate of bleed pump 210 pumping bleed material from the bioreactor). Generally, the bleed vessel is smaller than the bioreactor and can range anywhere from 250 mL and 2,000 L.

As used herein, the bleed vessel may be attached to another cell retention device. In the present disclosure, this device may be an ATF device 230. The bleed vessel may also incorporate two external pumps. One pump is connected to the permeate line of the ATF device and is referred to as “Harvest 2” pump (e.g., second harvest pump 240) in the present disclosure. The second pump is connected to the bottom of the bleed vessel and is referred to as “Bleed 2” pump (e.g., second bleed pump 250). In the present disclosure, the volumetric flowrates of the Harvest 2 plus Bleed 2 pumps equal that of the Bleed 1 pump rate in order to maintain the bleed vessel volume. In other words, the flowrate of the first bleed pump coupled to the bioreactor equals the flowrate of the second harvest pump coupled to the second ATF device plus the second bleed pump coupled to the bleed vessel. The Harvest 2 and Bleed 2 pumps can vary in flowrate as long as their combined rate is equal to Bleed 1 to maintain Bleed Tank volume.

As previously mentioned, in accordance with one or more features of the present disclosure, the bioreactor system 100, and in particular, the bleed recovery system 200, includes a second cell retention device or ATF device 230 coupled to the bleed vessel 220 so that the bleed material pumped into the bleed vessel 220 is further processed to separate product of interest from the spent material, which is discarded. Thus arranged, product of interest can be recovered, which would have otherwise been lost.

In use, as will be appreciated by one of ordinary skill in the art, the perfusion cell culture in the bioreactor 110 is monitored and maintained for optimal growth and production performance. The perfusion cell culture in the bioreactor 110 is monitored and maintained by any suitable mechanisms now known or hereafter developed. Any parameters that are monitored and controlled are known in the art. These optimized parameters that are controlled can include DO, pH, temperature, nutrients, and any other parameters in the art that are known to benefit cell culture. In this particular embodiment, an optimal Harvest 1 rate (e.g., rate of depositing product of interest within the first harvest tank 134) and Media In rate (e.g., rate of pumping new medium into the bioreactor) are used to maintain a healthy producing cell culture in the bioreactor 110. In this particular embodiment, an ATF device 120 is used as a cell retention device on the bioreactor 110. The product of interest collected from the Harvest 1 pump (e.g., harvest pump 132) may be used for further downstream processing.

In accordance with one or more features of the present disclosure, the Bleed 1 pump (e.g., the bleed pump 210) feeds culture from the bioreactor 110 into the bleed vessel 220. The bioreactor 110 can be anywhere between 250 mL and 25,000 L in volume. The bleed vessel 220 can be anywhere between 250 mL and 2,000 L in volume. As illustrated, in one embodiment, the bleed vessel 220 may be a sedimentation style vessel that does not use an agitation source. The rate of Bleed 1 (e.g., the flowrate of bleed pump 210) can be anywhere between 0.01 and 0.5 VVD of the bioreactor 110.

The Bleed 1 pump (e.g., the bleed pump 210) can be controlled by either offline or online measurements of VCD in order to maintain the VCD target. The VCD target that is maintained can be anywhere between 10e6 cells/mL and 300e6 cells/mL.

In accordance with one or more features of the present disclosure, the bleed vessel 220 is connected to a second cell retention device 230. As illustrated, in one embodiment, the second cell retention device 230 may be a second ATF device. In various embodiments, the second ATF device 230 may be coupled to the bleed vessel 220 on a side of the bleed vessel 220 (as illustrated in FIG. 1). Alternatively, the bleed vessel 220 may be coupled (or extend through) a top surface of the bleed vessel 220 (as illustrated in FIG. 2). In either event, as illustrated, the connection of the second ATF device 230 may be positioned far enough from the bottom of the bleed vessel 220 to prevent agitation of the settled cells. In use, during the operation of the second ATF device 230, the connection should be below the liquid level.

In some embodiments, the rate in which the bleed vessel material passes in and out of the second ATF device 230, known as ATF rate, can be anywhere between 0.1 LPM and 80 LPM. In one particular embodiment, the ATF rate equals the highest flow rate that does not disturb the settled cells.

In accordance with one or more features of the present disclosure, a second harvest pump 240 may be coupled to the permeate side of the second ATF device 230 to collect product of interest from the bleed vessel 220 that would otherwise or normally be lost in a perfusion cell culture process. In use, the Harvest 2 flow rate (e.g., flowrate of the second harvest pump 240) may be at or below one-twentieth the flowrate of the second ATF 230 coupled to the bleed vessel 220. The material collected from the Harvest 2 pump (e.g., second harvest pump 240) will be collected and deposited in a vessel referred to as Harvest 2 (e.g., a second harvest tank 242). This vessel can range anywhere between 250 mL and 2,000 L and the material collected in this vessel may be used for further downstream processing.

In use, in accordance with one or more features of the present disclosure, the Bleed 2 pump (e.g., second bleed pump 250) removes contents from the bleed vessel 220 and directs it into waste (e.g., waste tank 252) The Bleed 2 rate (e.g., flowrate of the second bleed pump 250) may be equal to the difference in the Bleed 1 rate (e.g., the flowrate of the first bleed pump 210) and the Harvest 2 rate (e.g., the flowrate of the second harvest pump 240) such that the bleed vessel volume is maintained. In one embodiment, the relationship of pump flow rates are as follows: Media In (e.g., rate of media pump 116 or flowrate of new medium being pumped into the bioreactor 110) equals the Bleed 1 rate (e.g., the flowrate of the first bleed pump 210 pumping bleed material out of the bioreactor 110) plus the Harvest 1 rate (e.g., flowrate of the first harvest pump 132 pumping product of interest into the first harvest tank 134) and the Bleed 1 rate (e.g., flowrate of the first bleed pump 210 pumping bleed material out of the bioreactor 110) equals the Bleed 2 rate (e.g., flowrate of the second bleed pump 250 pumping spent material out of the bleed vessel 220) plus the Harvest 2 rate (e.g., the flowrate of the second harvest pump 240 pumping product of interest out of the bleed vessel 220 and into the second harvest tank 242). These relationships are preferably maintained in order to keep working volumes constant in each vessel 110, 220.

In accordance with one or more features of the present disclosure, an example method of use is disclosed. In use, a perfusion cell culture using a CHO cell line producing a monoclonal antibody of interests is cultured in a 200 L single-use bioreactor 110. An ATF device 120 is used as a cell retention device for the production bioreactor. The ATF rate is set to 17 LPM and the Harvest 1 rate (e.g., flowrate of harvest pump 132) is 1 VVD or 200 L/day. The Bleed 1 rate (e.g., flowrate of the first bleed pump 210) is set to 0.1 VVD or 20 L/day to maintain a VCD target of 40e6 cells/mL. Therefor the Media in rate (e.g., rate of media pump 116 or flowrate of new medium being pumped into the bioreactor 110) is 1.1 VVD. The bleed vessel collection material from the Bleed 1 pump (e.g., bleed pump 210) fills at a rate of 20 L/day. The sedimentation style tank (e.g., bleed vessel 220) is a 200 L tank that uses a second ATF device 230 as a cell retention device. The connection of the second ATF device 230 is on the side of the sedimentation style tank preferably around the probe belt. Once the bleed material reaches the connection of the second ATF device 230, the ATF will run at 8 LPM. The Harvest 2 pump (e.g., the flowrate of the second harvest pump 240) on the ATF pump operates at 18 L/day and the Bleed 2 pump (e.g., flowrate of second bleed pump 250) operates at 2 L/day. This maintains the bleed vessel volume. The material collected from the Harvest 2 pump (e.g., second harvest tank 242) will be further processed downstream.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

Various features, aspects, or the like of a vessel or process system may be used independently of, or in combination, with each other. It will be appreciated that a vessel and/or system as disclosed herein may be embodied in many different forms and should not be construed as being limited to the illustrated embodiments of the figures, such as described herein. Rather, these embodiments are provided so that this disclosure will convey certain aspects of a vessel and/or process system formed in accordance with various principles of the present disclosure to those skilled in the art.

It should be understood that, as described herein, an “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more embodiments of concepts or features together in a single embodiment of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one embodiment can be used separately, or with one or more other features to yield a still further embodiment. Thus, it is intended that the present subject matter covers such modifications and variations as come within the scope of the appended claims and their equivalents.

In view of the above, it should be understood that the various embodiments illustrated in the figures have several separate and independent features, which each, at least alone, has unique benefits which are desirable for, yet not critical to, the presently disclosed vessel, system, and associated method. Therefore, the various separate features described herein need not all be present in order to achieve at least some of the desired characteristics and/or benefits described herein. Only one of the various features may be present in a vessel or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, throughout the present disclosure, reference numbers are used to indicate a generic element or feature of the disclosed embodiment. The same reference number may be used to indicate elements or features that are not identical in form, shape, structure, etc., yet which provide similar functions or benefits. Additional reference characters (such as letters, as opposed to numbers) may be used to differentiate similar elements or features from one another.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for harvesting product of interest from a bleed material of a perfusion bioreactor, the system comprising:
a bioreactor arranged and configured to store a medium including a product of interest;
a first cell retention device coupled to the bioreactor via tubing, the first cell retention device arranged and configured to separate the product of interest from the medium;
a first harvest pump coupled to the first cell retention device via tubing to transfer the product of interest from the first cell retention device to a first harvest tank; and
a bleed recovery system coupled to the bioreactor via tubing, the bleed recovery system arranged and configured to receive the bleed material including the product of interest, wherein the bleed recovery system includes:
a first bleed pump operatively coupled to the bioreactor via tubing;
a bleed vessel connected the first bleed pump, the bleed vessel arranged and configured to receive the bleed material including the product of interest from the first bleed pump;
a second cell retention device coupled to the bleed vessel via tubing, the second cell retention device arranged and configured to receive the product of interest;
a second harvest pump coupled to the second cell retention device via tubing to transfer the product of interest from the second cell retention device to a second harvest tank; and
a second bleed pump connected to the bleed vessel to transfer the medium to a bleed waste tank.

2. The system of claim 1, wherein the bleed vessel is a sedimentation style bleed vessel.

3. The system of claim 2, wherein the second cell retention device is coupled to one of a side portion or a top portion of the sedimentation style bleed vessel.

4. The system of claim 3, wherein the second bleed pump is connected to a bottom of the sedimentation style bleed vessel.

5. The system of claim 1, wherein the first cell retention device and the second cell retention device are one of an alternating tangential flow (ATF) filtration device or a tangential flow filtration (TFF) device.

6. The system of claim 1, wherein a flowrate of the first bleed pump equals a flowrate of the second bleed pump plus a flowrate of the second harvest pump.

7. The system of claim 1, wherein a flowrate of the first bleed pump is semi-continuous, constantly continuous, or dynamically continuous.

8. The system of claim 1, further comprising a fresh media tank including fresh medium including product of interest and a media pump arranged and configured to pump fresh medium into the bioreactor.

9. The system of claim 8, wherein the media pump includes a flowrate equal the first harvest pump plus first bleed pump.

10. The system of claim 8, wherein a flowrate of the first bleed pump equals a flowrate of the media pump minus a flowrate of the first harvest pump.

11. The system of claim 1, wherein a flowrate of the first harvest pump is semi-continuous, constantly continuous, or dynamically continuous.

12. The system of claim 1, a flowrate of the first bleed pump is increased or decreased to maintain a target cell density within the bioreactor.

13. The system of claim 1, wherein a flowrate of the second bleed pump and the second harvest pump are operated semi-continuously, constantly continuous, or dynamically continuous.

14. The system of claim 1, wherein a flowrate of the second bleed pump is equal to a flowrate of the first bleed pump minus a flowrate of the second harvest pump once a level of the bleed material in the bleed vessel reaches an inlet of the second cell retention device.
